(19) United States
(12) Patent Application Publication
(10) Pub. No.: US 2021/0196816 A1
(43) Pub. Date: Jul. 1, 2021

(54) ALGAE COMPRISING THERAPEUTIC AND/OR NUTRITIONAL AGENTS

(71) Applicant: TomAlgae CVBA, Nevele (BE)

(72) Inventors: Viktor Chepurnov, Gent (BE); Marc Temmerman, Zwijnaarde (BE); William Van Der Riet, Hoogland (NL)

(21) Appl. No.: 16/335,114

(22) PCT Filed: Sep. 20, 2017
(86) PCT No.: PCT/EP2017/073834

§ 371 Date: Mar. 20, 2019

(30) Foreign Application Priority Data
Sep. 21, 2016 (GB) 1616087.1

(51) Int. Cl. A61K 39/385; A61K 36/04; C12N 1/12; A23L 17/60; A23L 33/105; A23K 10/30

(52) U.S. Cl. CPC A61K 39/385; A61K 36/04; C12N 1/12; A61K 2039/522; A23L 33/105; A23K 10/30; A23L 17/60

(57) ABSTRACT

An alga, particularly a diatom, comprising one or more selected from the group consisting of: a therapeutic agent (such as an immunogenic agent; antibody; anti-microbial agent; anti-parasitic agent and appetite promoter); an exogenous nutritional agent; and an enhanced level of an endogenous nutritional agent, used as a diet enhancer, a drug delivery device, a vaccine delivery device and/or an animal feed, or for use for use in therapy, a method of preparing an alga comprising: providing a dehydrated alga; and rehydrating the alga in the presence of a therapeutic agent or nutritional agent, and related kits.

The present invention relates to an alga, in particular a diatom alga, comprising one or more therapeutic agents, one or more exogenous nutritional agents and/or an enhanced level of one or more endogenous nutritional agents, the use of the same as a diet enhancer, a drug delivery device, a vaccine delivery device and/or an animal feed, the same for use in treatment, methods for making the same, and related kits.

Algae is the term generally accepted for a large diverse group of eukaryotes having chlorophyll as their primary photosynthetic pigment. Algae are the primary producers in aquatic ecosystems, which are the starting point of the food chain or web. Around 3% of algae are macroalgae (e.g. seaweed) and the remaining 97% are microalgae.

Microalgae are used in human nutritional supplements and animal feeds. Microalgae may also be used in the production of fertilizers, pigments and biofuels, as well as in wastewater treatments.

The addition of microalgae to animal feed has the ability to enhance feed nutritional content, thus improving their effect in animal health. Microalgae such as microphytes constitute the basic foodstuff for numerous aquaculture species, and one application of microalgae feeds is in aquaculture. To be suitable for aquaculture, microalgae must have specific characteristics, for example, be of an appropriate size for ingestion, have a rapid growth rate, be stable in culture, and have a good nutrient profile.

There is increasing dependence on aquaculture to provide seafood as natural habitats are deteriorated, and natural fish populations are unsustainably fished by an increasing global human population. Therefore, a global aquaculture industry has developed exponentially. The value of the aquaculture industry, as with other industries, depends on the quality and quantity of the product that can be produced. Aquaculture operators therefore seek to optimise the feed to try to increase the quality and quantity of the produced seafood. For example, many seafood hatcheries use a feed comprising live microalgal populations, which is optionally enriched in a formulation with soluble or insoluble chemical or other factors by admixture.

However, when such enriched feed is added to an aquaculture tank, in particular to a large aquaculture tank such as one containing thousands of litres of water, dosing of the enrichment factors becomes difficult because the factors are often not co-localised with the feed.

Dosing and delivery of nutrients is also a problem in animal feeds for terrestrial animals. For example, enrichment factors are often unevenly distributed among the feed or may become unevenly distributed during transit and storage.

Additionally, live microalgal cultures are not reliable in terms of their size or the quality of the algae contained, and are subject to colonisation with non-target algae or other species. This colonisation may diminish or dilute the target algae, and so cause damage to the animal population, directly or indirectly.

Thus, dried food may be used, for example, in the form of dried algae, which is subject to rigorous quality control. This ensures, firstly, the supply of an adequate quantity of feed and, secondly, the quality and suitability of the feed.

However, there is a need for an improved or alternative dried food for use in animal feeds for example, for terrestrial animal husbandry and aquaculture.

The use of dried feed facilitates the combination of the feed with the enrichment factors, for example, in the form of a pellet. However, such pellets are not suitable for feeding to certain animals that feed on small particles such as filter feeders and shrimp.

The concentration of animals in close proximity in the relatively limited space of animal enclosures or volume of aquaculture tanks can result in diseases spreading quickly amongst the animal population.

For example, in aquaculture, infection by bacteria or viruses, or infestation of lice, such as sea lice, can result in the loss of entire populations.

The farming and aquaculture industries therefore routinely administer therapeutic agents for prophylaxis or treatment of diseases. Administration to animals may, for example, be by the following routes (as appropriate to the species): topical application; immersion, i.e. by adding the soluble or insoluble agent to the culture water; injection directly into the animal; or in-feed.

However, injection and topical application is distressing for the animal and labour intensive. For example, in aquaculture, immersion application addresses this issue, but requires higher dosing of the water to ensure that the animals receive an adequate dose.

Providing a therapeutic agent in-feed in an admixed powdered formulation is no better than immersion application, as the admixed formulation dissociates. The use of dried feed facilitates the combination of the feed with the therapeutic agents, for example, in the form of a pellet. This concentrates the dose of the therapeutic agent in a form that the animals are motivated to seek out and consume, thus lowering the dosage of the culture water. However, such pellets are not suitable for feeding to animals that feed on small particles such as filter feeders and shrimp.

There is thus a need for means to reliably dosing animal feed with therapeutic or nutritional agents. For example, there is a need for means to reliably dose aquaculture feed with therapeutic or nutritional agents for use with species that feed on small particles, such as filters feeders, fish fry and shrimp.

Accordingly, a first aspect of the invention provides an alga comprising one or more selected from the group consisting of: a therapeutic agent; an exogenous nutritional agent; and an enhanced level of an endogenous nutritional agent.

In embodiments of the invention, the alga is not transgenic, genetically modified, or otherwise molecular biologically-modified, e.g. by way of recombinant DNA technology, to express the therapeutic agent, the exogenous nutritional agent or the enhanced level of the endogenous nutritional agent.

In embodiments of the invention, the alga is transgenic, genetically modified, or otherwise molecular biologically-modified, e.g. by way of recombinant DNA technology, to express the therapeutic agent, the exogenous nutritional agent or the enhanced level of the endogenous nutritional agent.

In embodiments of the invention, the therapeutic agent, the exogenous nutritional agent or the enhanced level of the endogenous nutritional agent is added to/enriched in the algae by both molecular biological (e.g. transgenics, genetic modification) and non-molecular biological means (e.g. by loading the alga according the present invention).

While the invention provides a single alga, the invention will typically take the form of a population comprising a plurality of such algae. Thus, reference to an alga will in most cases also refer to algae, and reference to algae will in most cases also refer to a single alga.

Embodiments of this aspect of the invention provide an alga comprising 2 or more, 3 or more, 4 or more, or 5 or more therapeutic agents.

By “nutritional agent” we refer to a substance that is additive to the nutritional value of the alga to the target feeder. An embodiment of the invention provides an alga comprising an exogenous nutritional agent. That is, the alga is loaded with a nutritional agent that is not normally present in the alga.

Embodiments of this aspect of the invention provide an alga comprising 2 or more, 3 or more, 4 or more, or 5 or more exogenous nutritional agents.

An embodiment of the invention provides an alga comprising an enhanced level of an endogenous nutritional agent. That is, the alga is loaded with a substance that is already present in the alga to enhance the level of the substance.

Embodiments of this aspect of the invention provide an alga comprising an enhanced level of 2 or more, 3 or more, 4 or more, or 5 or more endogenous nutritional agents.

Thus, the alga is loaded with a therapeutic or nutritional agent, and so the agent is co-localised with the alga. When the alga is used as an animal feed, the animal is motivated to consume the feed, and so inevitably consumes the therapeutic or nutritional agent. This advantageously provides a mechanism to selectively target the therapeutic or nutritional agent in the subject requiring therapy or enhanced nutrition. This mode of delivery reduces the dosage required using prior art methods, such as immersion application via a tank containing a large volume of aquaculture water.

The alga also provides some protection for the agent against chemical and/or enzymatic degradation in the stomach of the animal, thus increasing the bioavailability of the agent and reducing the dosage required for a given effect.

In some embodiments, the alga may be fed directly to a target animal. In alternative embodiments, the alga may be fed to an intermediate animal, which is turn is used as a feed for the target animal.

For example, the alga may be fed to an Artemia, which is subsequently fed to a fish.

Further aspects of the invention provide an alga comprising two or three of: a therapeutic agent; an exogenous nutritional agent; or an enhanced level of an endogenous nutritional agent. Embodiments of these aspects of the invention provide an alga comprising one or more of the following:
2 or more, 3 or more, 4 or more, or 5 or more therapeutic agents;
2 or more, 3 or more, 4 or more, or 5 or more exogenous nutritional agent; and/or
2 or more, 3 or more, 4 or more, or 5 or more endogenous nutritional agents.

Thus, the invention provides a means to concentrate combinations of one or more therapeutic and/or one or more nutritional agents.

In embodiments of all aspects of the invention, the alga is a microalga, preferably a diatom.

By “diatom” we mean alga of the division Bacillariophyta.

In contrast to other algae that have cellulose cell wall, diatoms have cell walls comprising hydrated silicon dioxide called frustules, making them robust enough to endure dehydration and/or lyophilisation while maintaining adequate cell integrity.

In some embodiments, the diatom is a centric diatom belonging to the class Coscinodiscophyceae (centric diatoms). In some embodiments, the diatom belongs to the subclass Thalassiosirophycidae. In some embodiments, the diatom belongs to the order Thalassiosirales. Preferably, the diatoms belong to a genus selected from the group consisting of: Thalassiosira, Cyclotella and Skeletonema.

The classification of diatoms is as proposed by Round et al. (1990) Diatoms: Biology and Morphology of the Genera, Cambridge University Press, UK: pp 125-129.

In some embodiments, the alga belongs to the genus Cyclotella. Preferably, the alga is Cyclotella meneghiniana. C. meneghiniana advantageously is rich in polyunsaturated fatty acids, omega-3 fatty acids, and is readily digestible by organisms that feed on small parts, such as juvenile shrimp. In alternative embodiments, the alga is C. cryptica.

In alternative embodiments, the alga belongs to the genus Thalassiosira. Preferably, the alga is T. weissflogii or T. pseudonana.

In alternative embodiments, the diatoms belong to the order Centrales, Melosirales or Coscinodiscophyceae. The diatoms may belong to a genus selected from the group consisting of: Odontella, Melosira and Coscinoliscus.

It has been found that diatoms, for example diatoms of the genus Cyclotella, are particularly suitable for being loaded with therapeutic and/or nutritional agents. The presence of the silica cell wall (frustule) makes them more robust than other algae and thus they can be subjected to rehydration, dehydration and lyophilisation steps whilst critically maintaining cell integrity.

In embodiments of the invention, the therapeutic agent is selected from the group consisting of: immunogenic agent; antibody; anti-microbial agent; anti-parasitic agent and appetite promoter.

In embodiments of the invention, the therapeutic agent may be a naturally occurring, synthetic, or semi-synthetic material (e.g., compounds, fermentates, extracts, cellular structures) capable of eliciting, directly or indirectly, one or more physical, chemical and/or biological effects.

In embodiments of the invention, the therapeutic agent may be capable of preventing, alleviating, treating and/or curing abnormal and/or pathological conditions of a living body, such as by destroying a parasitic organism, or by limiting the effect of a disease or abnormality. Depending on the effect and/or its application, the therapeutic agent may be a pharmaceutical agent (such as for prophylaxis or treatment), a diagnostic agent and/or a cosmetic agent, and includes, without limitation, vaccines, drugs, prodrugs, affinity molecules, synthetic organic molecules, hormones, antibodies, polymers, enzymes, low molecular weight molecules proteinaceous compounds, peptides, vitamins, steroids, steroid analogues, lipids, nucleic acids, carbohydrates, precursors thereof, and derivatives thereof.

In embodiments of the invention, the therapeutic agents in the present invention may include vaccines, antibiotics, affinity molecules, synthetic organic molecules, polymers, low molecular weight proteinaceous compounds, peptides, vitamins, steroids, steroid analogues, lipids, nucleic acids, carbohydrates, precursors thereof, and derivatives thereof. The bioactive agent may also be a pesticide.

Where the therapeutic agent is a vaccine, the vaccines may also be delivered as part of immune-stimulating complexes, conjugates of antigens with cholera toxin and its B subunit, lectins and adjuvants.

The therapeutic agent may comprise a virus-like particle. The virus-like particle may be used to deliver a nucleotide-based, or peptide based therapeutic, such as a vaccine.

The therapeutic agent may be an immunogen, i.e., a material capable of mounting a specific immune response in an animal. Examples of immunogens include antigens and vaccines. For example, immunogens may include immunogenic peptides, proteins or recombinant proteins, including mixtures comprising immunogenic peptides and/or proteins and bacteria (e.g., bacterins); intact inactive, attenuated, and infectious viral particles; intact killed, attenuated, and infectious prokaryotes; intact killed, attenuated, and infectious protozoans including any life cycle stage thereof, and intact killed, attenuated, and infectious multicellular pathogens, recombinant subunit vaccines, and recombinant vectors to deliver and express genes encoding immunogenic proteins (e.g., DNA vaccines).

In embodiments of the invention, the nutritional supplement may include one or more of protein, carbohydrate, water-soluble vitamin (e.g., vitamin C, a B-complex vitamin), fat-soluble vitamins (e.g., vitamins A, D, E, K), minerals and herbal extracts.

Thus, in embodiments of the invention the therapeutic agent or nutritional agent is selected from the group consisting of: protein; carbohydrate; nucleotide; lipid; and steroid. In embodiments of the invention the endogenous or exogenous nutritional agent may be an omega-3 polyunsaturated fatty acid, pigments, a vitamin, a steroid, or an amino acid. The pigment may be a carotenoid pigment such as astaxanthine and fucoxanthin. The polyunsaturated fatty acid may be eicosapentaenoic acid (EPA) or docosahexaenoic acid (DHA). The vitamin may be vitamin C. The amino acid may be an essential amino acid. This list is not exhaustive and it would be appreciated that there may be other suitable endogenous or exogenous agents that could be loaded into algae in accordance with the present invention.

Taking the example of Cyclotella, an example of an exogenous nutritional agent (i.e. a carotenoid that is not normally present in the alga) that can be introduced into the alga is the carotenoid astaxanthine is, whereas an example of an endogenous nutritional agent (i.e. a carotenoid that is already present in the alga) that can be enhanced in the alga is fucoxanthin.

In general, it will be appreciated the therapeutic or nutritional agent may be any agent that there is a desire to deliver to the animal that is loadable to the alga. The therapeutic or nutritional agent may be 150 nm or less in diameter, 100 nm or less in diameter, 85 nm or less in diameter, or 75 nm or less in diameter. In an embodiment, the therapeutic or nutritional agent may be 50-150 nm or less in diameter. In an embodiment, the therapeutic or nutritional agent may be larger than 150 nm in diameter

In some embodiments of the invention, the therapeutic or nutritional agent is loaded to the pores in the frustule of the algae and/or loaded via the valve.

In embodiments of the invention, the therapeutic or nutritional agent is substantially contained within the alga cytoplasm or sub-cellular compartment. In some embodiments, the therapeutic or nutritional agent is substantially contained within the cell membrane.

In embodiments of the invention, the immunogenic agent is derived from a pathogen. Optionally, the immunogenic agent comprises an effective amount of a purified antigen.

In specific embodiments if the invention, the therapeutic or nutritional agent is present in the alga at a level of at least 5 μg, at least 10 μg, at least 20 μg, at least 50 μg, at least 100 μg, at least 200 μg, at least 500 μg, at least 1 mg, at least 10 mg, at least 20 mg, or at least 50 mg of agent per g (dry weight) of algae.

A further aspect of the invention provides a use of an alga according to the invention as a diet enhancer, a drug delivery device, a vaccine delivery device, or more generally an animal feed.

A further aspect of the invention provides a diet enhancer, a drug delivery device, a vaccine delivery device and/or an animal feed comprising an alga according to the invention.

In some embodiments of the invention, particularly where the alga is loaded with a therapeutic agent, the organic components of the alga cell may be partially or fully removed such that the cell has minimal or no nutritional value, but can still be used for delivery of the therapeutic or nutritional agent.

A further aspect of the invention provides an alga according to the invention for use in therapy.

A further aspect of the invention provides an alga according to the invention for use in the treatment of viral, bacterial or parasitic infection/infestation.

A further aspect of the invention provides a method of treatment, comprising administering to an animal an alga comprising a therapeutic agent according to the invention, preferably wherein the therapeutic agent is suitable for use in the treatment of viral, bacterial or parasitic infection/infestation.

For example, where the animal is a fish, the alga may be for use in treating the following infections: infectious pancreatic necrosis (IPNV); pancreas disease (PDV); infectious salmon anaemia (ISAV); infectious hematopoietic necrosis (VHSV); viral nervous necrosis; iridoviral disease (RSIV); channel catfish virus disease (CCV); spring viremia of carp (SVCV); grass carp haemorrhage disease (GCHDV); Vibrio spp., Listonella anguillarum; Vibrio harveyi; Vibrio salmonicida; Moritella viscosa; Aeromonas salmonicida subsp. salmonicida; Aeromonas salmonicida; Yersinia ruckeri; Piscirickettsia salmonis; Flavobacterium branchiophilum; Flavobacterium psychrophilum; Edwardsiella ictaluri; Edwardsiella tarda; Renibacterium salmoninarum; Lactococcus garvieae; Photobacterium subspecies piscicida; Streptococcus iniae; Streptococcus phocae; Piscirickettsia salmonis; Flavobacterium columnare; Paramoeba spp. (Amoebic gill disease); Cryptobia salmositica; Ichthyobodo spp.; Ichthyophthirius multifilis (White spot disease); Cryptocaryon irritans; Trichondina spp.; Tetramicra brevifilum; Pleistophora anguillarum; Nucleospora salmonis; Myxobolus cerebrialis (whirling disease); Tetracapsula bryosalmonae (proliferative kidney disease; PKD); Kudoa thyrsites; Gyrodactylus spp.; Dactylogyrus spp.; Benedinia spp.; Neobenedinia spp.; Eubothrium spp.; Lepeophtheirus salmonis; or Caligus spp.

For example, where the animal is a shrimp, the alga may be for use in treating the following infections: infectious hypodermal and hematopoietic necrosis virus (IHHNV); yellow head virus (YHV); taura syndrome virus (TSV); infectious myonecrosis (IMN); white spot syndrome virus (WSSV); or Vibrio spp.

For example, where the animal is a terrestrial animal, the alga may be for use in treating the following infections: African horse sickness; African swine fever; anaplasmosis; bluetongue; bovine spongiform encephalopathy; bovine tuberculosis; brucellosis; chronic wasting disease; classical swine fever (hog cholera); contagious bovine pleuropneumonia; contagious equine metritis; cysticercosis; equine infectious anemia (EIA); equine piroplasmosis; foot and mouth disease; fowl typhoid; lumpy skin disease; Newcastle disease; notifiable avian influenza; pseudorabies (Aujeszky's disease); pullorum disease; rift valley fever; rinderpest; sheep and goat pox; swine vesicular disease; trichinellosis; Venezuelan equine encephalomyelitis; vesicular stomatitis; mastitis; porcine circovirus (e.g. porcine circovirus type 2); porcine reproductive and respiratory syndrome; clostridial disease (e.g. black disease, blackleg, malignant oedema, enterotoxemia, tetanus and botulism).

It will be appreciated that these lists are not exhaustive and that the alga of the present invention may be provided for use in the treatment of any known disease or condition, including metabolic syndromes and nutritional diseases which require supplementation.

In an embodiment of the invention, the alga is administered to an animal by direct oral administration or immersion. In the present context, direct oral administration means directly placing the alga in the mouth or mouth parts of the animal. This contrasts with immersion administration, which relies on the animal finding the alga in the culture water and allowing the alga to enter its mouth or mouthparts.

In another embodiment of the invention, the alga is administered to an animal via another intermediate animal. For example, the alga may be fed to an intermediate animal such as an Artemia, which in turn is administered, typically fed, to another animal.

A further aspect of the invention provides an animal feed comprising the alga according to the invention.

A further aspect of the invention provides a vaccine composition comprising the alga according to the invention.

A further aspect of the invention provides a medicament composition comprising the alga according to the invention.

A further aspect of the invention provides a composition comprising the algae according to the invention formulated with one or more pharmaceutically acceptable ingredients, adjuvants and/or excipients.

A further aspect of the invention provides a method of preparing an alga comprising: providing a dehydrated alga; and rehydrating the alga in the presence of a therapeutic agent or nutritional agent, thus loading the alga with the agent.

Loading the algae with the agent is thus different to merely mixing the algae with a therapeutic agent or nutritional agent to form a wet paste, or mixing granulated dry algae with a dry therapeutic agent or nutritional agent to form a dry admixture.

In embodiments of the invention, the alga is not transgenic, genetically modified, or otherwise molecular biologically-modified, e.g. by way of recombinant DNA technology, to express a therapeutic agent, an exogenous nutritional agent or an enhanced level of the endogenous nutritional agent.

In embodiments of the invention, the alga is transgenic, genetically modified, or otherwise molecular biologically-modified, e.g. by way of recombinant DNA technology, to express an therapeutic agent, an exogenous nutritional agent or an enhanced level of the endogenous nutritional agent. The expressed therapeutic agent, exogenous nutritional agent or enhanced level of the endogenous nutritional agent may be the same or different to the loaded therapeutic agent, exogenous nutritional agent or enhanced level of the endogenous nutritional agent.

In embodiments of the invention, the therapeutic agent or nutritional agent is produced in an alga, which may optionally be transgenic, genetically modified, or otherwise molecular biologically-modified, e.g. by way of recombinant DNA technology, to express an therapeutic agent, an exogenous nutritional agent or an enhanced level of the endogenous nutritional agent, then loaded into another alga that is not transgenic, genetically modified, or otherwise molecular biologically-modified, e.g. by way of recombinant DNA technology, to express a therapeutic agent, an exogenous nutritional agent or an enhanced level of the endogenous nutritional agent.

The method may further comprise dehydrating or lyophilising the rehydrated (loaded) alga. The dehydrated/lyophilised loaded algae can thus be stored as a dry product and rehydrated when required for use.

In embodiments of the invention, the rehydrating comprises:
adding therapeutic agent or nutritional agent to water to form a rehydration composition;
agitating the rehydration composition; and
adding the alga to the composition under continued agitation.

Thus, the method according to the invention advantageously does not require growing the algae on a supplemented medium so they take up the therapeutic agent or nutritional agent.

Preferably, the agitation is blending. Blending ensures that the alga are sufficiently rehydrated, and that any air is expelled from the preparation.

In embodiments of the invention, the water used to rehydrate the alga is freshwater or seawater. The skilled person would select the most appropriate type of water depending on the particular application, for example, to match the aquaculture conditions for the species being farmed.

A further aspect of the invention, provides an animal feed comprising the alga according to the invention. In embodiments of the invention, the animal feed may consist only of the alga according to the invention. In other embodiments of the invention, the animal feed further comprises another source of nutrition.

In embodiments of the invention, the animal feed comprises loaded algae according to the invention and unloaded algae. The unloaded algae may be the same as the loaded algae, except that they are not loaded with a nutritional agent or a therapeutic agent.

Thus, the unloaded algae may optionally be rehydrated using a method comprising:
providing a dehydrated alga; and
adding the alga to agitated water, optionally freshwater or seawater.

A further aspect of the invention provides a method of preparing an animal feed comprising admixing the alga according the invention with feed material.

In embodiments of the invention, the feed mixture is milled into one selected from the group consisting of: meal type; pellets; and crumbles. Thus, the size of the feed mixture can be tailored to the feeder species.

In embodiments of the invention, the animal feed is prepared as a pellet and is optionally coated with an enteric polymer coating.

A further aspect of the invention provides a method of administering a therapeutic or nutritional agent to an animal, the method comprising administering to the animal the alga according to the invention by direct oral administration or immersion.

Direct oral administration advantageously reduces the amount of loaded algae required for dosing. Immersion administration advantageously reduces stress on the subject, reduces the technical expertise required to administer the agent, and enables dosing of animals for which direct oral administration is not possible, for example, when the subject is too small to handle.

A further aspect of the invention provides a delivery system for delivery of a therapeutic agent or nutritional agent to an animal comprising the algae according to the invention. The delivery system, such as a drug delivery system, provides a convenient, inexpensive means to dose animals using a form that the animal would be motivated to seek out and consume, since the system is recognised as food. This improves compliance and efficiency of nutritional or therapeutic treatment.

In embodiments of the invention the delivery system is a diet enhancer, a drug delivery device, a vaccine delivery device and/or an animal feed.

A further aspect of the invention provides a kit of parts for delivery of a therapeutic agent or nutritional agent to an animal, the kit comprising algae according to the invention and instructions for use.

In some embodiments, the kit comprises algae loaded with one or more therapeutic or nutritional agents in a dehydrated or lyophilised form and instructions for rehydration. In alternative embodiments, the kit comprises algae in a hydrated form, and may further comprise one or more preservatives.

The kit may comprise algae, preferably in a dehydrated form, one or more therapeutic and/or nutritional agents and instructions for rehydration of the algae to obtain algae loaded with one or more therapeutic or nutritional agents.

The dehydrated algae and one or more therapeutic or nutritional agents may be provided separately or mixed as one dry formulation ready to rehydrate.

A further aspect of the invention provides an animal treated with the alga according to the invention.

Specific embodiments of the invention will now be described with respect to the following, non-limiting examples in which:

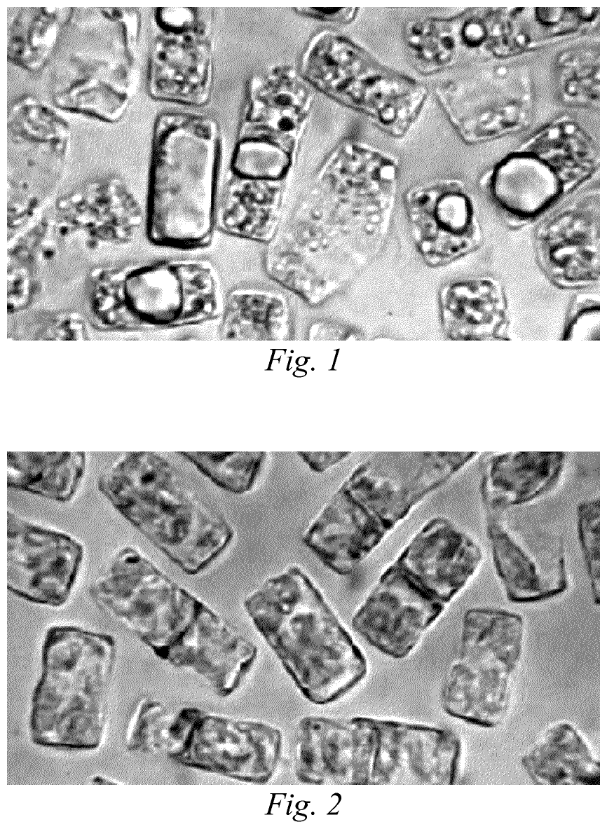

FIG. 1 is an image showing Cyclotella meneghiniana rehydrated in the presence of water and omega-3 DHA EE (docosahexaenoic acid ethyl esters) fish oil;

FIG. 2 is an image showing Cyclotella meneghiniana rehydrated in the presence of water with no oil;

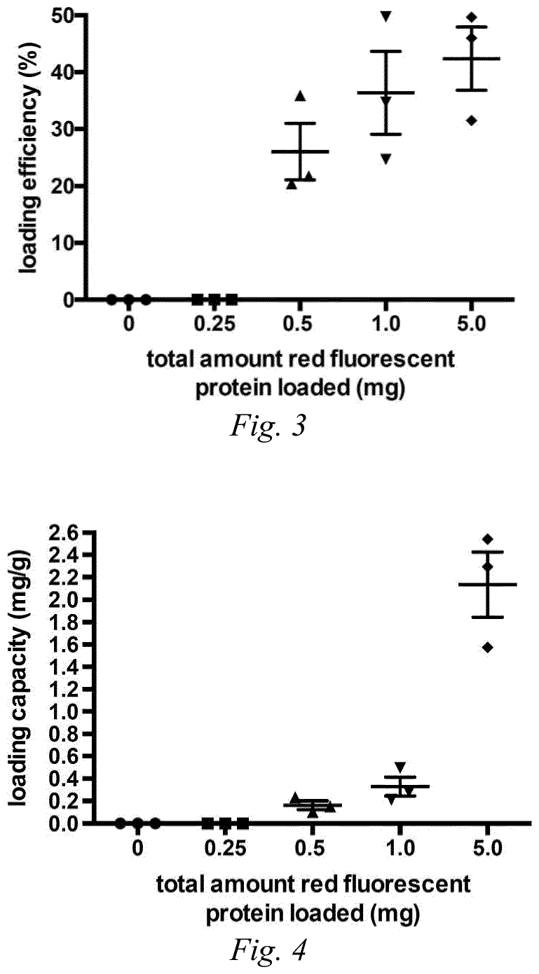

FIG. 3 shows the loading efficiency for algae rehydrated in the present of a recombinant protein at five different loading levels;

FIG. 4 shows the loading capacity for algae rehydrated in the present of a recombinant protein at five different loading levels;

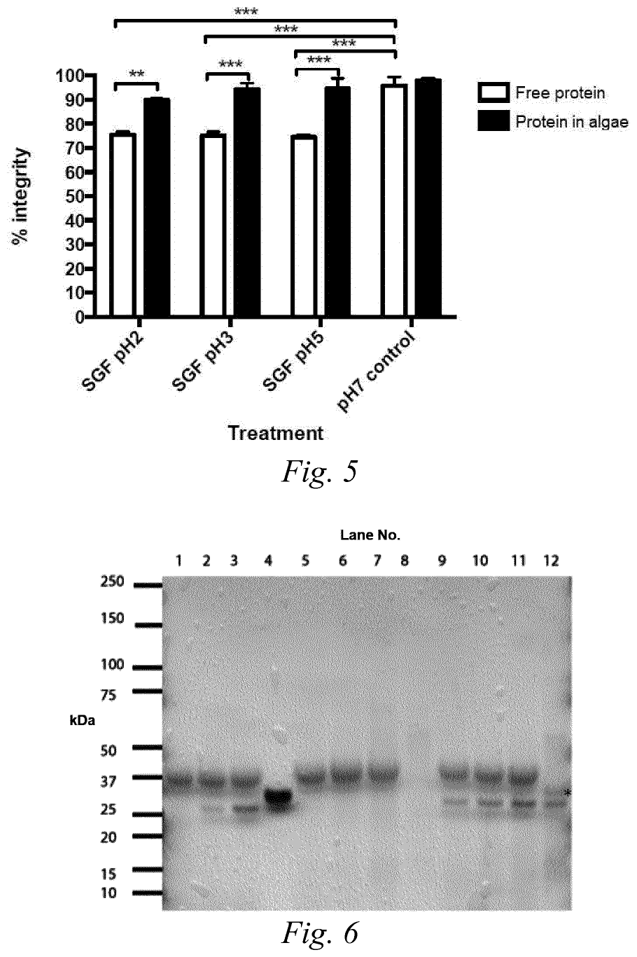

FIG. 5 shows percent integrity of recombinant red fluorescent protein (RFP) loaded into diatoms (“Protein in algae”) versus free rRFP (“Free protein”) incubated in simulated gastric fluid (SGF) at pH 2, 3 or 5, or a saline control pH 7 for 4 hours at 28° C. and at 100 rpm agitation. Bars represent average protein integrity of three independent experiments±SEM. ** denotes statistical significant to p<0.005 and *** denotes statistical significant to P<0.0005;

FIG. 6 shows an SDS-PAGE gel illustrating degradation of free rRFP, and release and degradation of rRFP loaded into diatoms, incubated in simulated gastric fluid (SGF) at pH 2, 3 and 5, and in a saline control pH 7, for 4 hours at 28° C. and 100 rpm agitation. Lanes 1-4: free rRFP; lanes 5-8: unloaded diatoms; lanes 9-12: rRFP loaded into diatoms; Lanes 1, 5 and 9: SGF at pH 2; lanes 2, 6 and 10: SGF at pH 3; lanes 3, 7 and 11: SGF at pH 5; lanes 4, 8 and 12: saline control at pH 7. * highlights non-degraded rRFP at approx. 28 kDa. RFP degradation products can bee seen at approximately 26 and 24 kDa, and pepsin from porcine gastric mucosa at approximately 35 kDa in lanes from SGF at pH 2, 3 and 5;

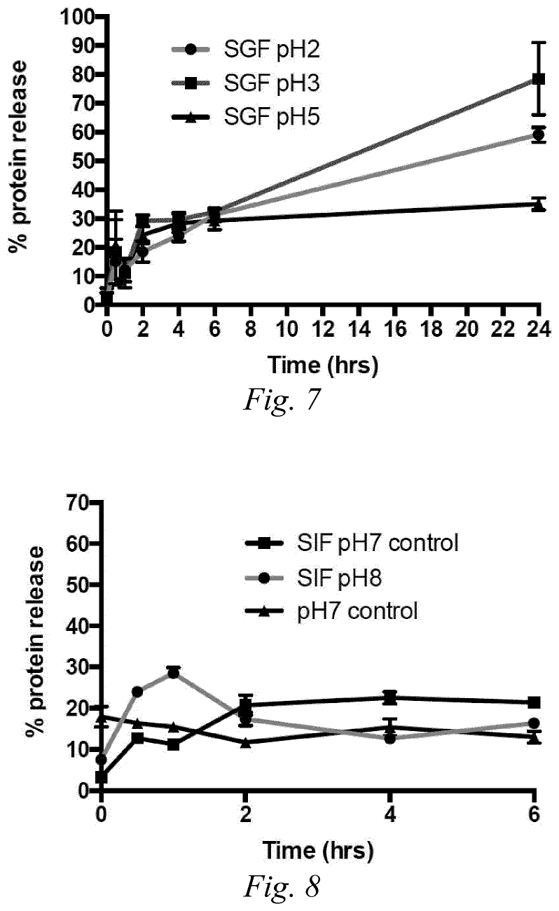

FIG. 7 shows percent release of rRFP loaded into diatoms and incubated in simulated gastric fluid (SGF) at pH 2, 3 and 5 for 0, 0.5, 1, 2, 4, 6 and 24 hours at 28° C. and 100 rpm agitation. Data points represent average protein release of three independent experiments±SEM;

FIG. 8 shows percent release of rRFP loaded into diatoms and incubated for 4 hours in SGF at pH 3, then in simulated intestinal fluid (SIF) at pH 8 or SIF at pH 7 (control; no enzymes) for 0, 0.5, 1, 2, 4, 6 and 24 hours at 28° C. and 100 rpm agitation versus that of rRFP-loaded diatoms incubated in a saline control pH 7 for the duration of the experiment. Data points represent average % protein release of three independent experiments±SEM;

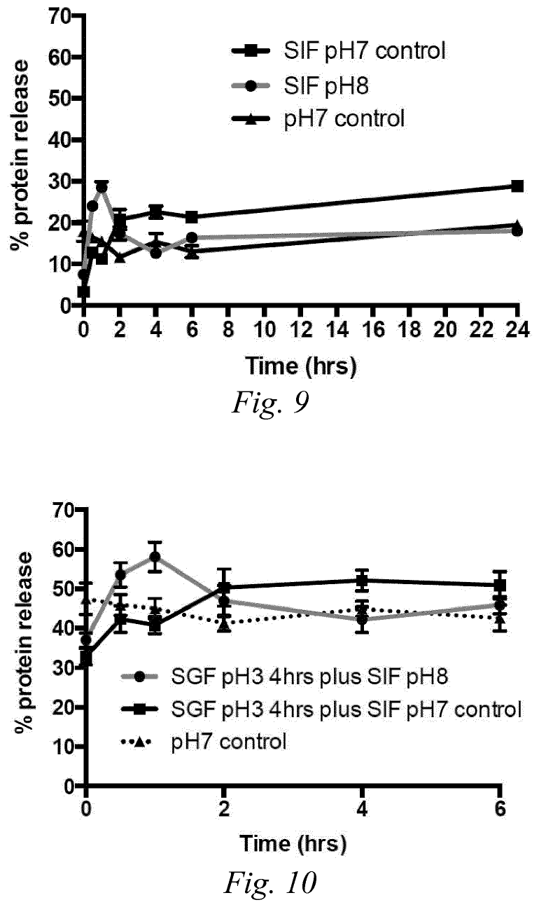

FIG. 9 shows percent release of rRFP loaded into diatoms and incubated for 4 hours in SGF at pH 3, then in SIF at pH 8 or SIF at pH 7 (control; no enzymes) for 0, 0.5, 1, 2, 4, 6 and 24 hours at 28° C. and 100 rpm agitation compared to percent release of rRFP from rRFP-loaded diatoms incubated in a saline control pH 7 for the duration of the experiment. Data points represent average % protein release of three independent experiments±SEM;

FIG. 10 shows total cumulative percent release of rRFP loaded into diatoms and incubated for 4 hours in SGF pH 3, then in SIF at pH 8 or SIF at pH 7 (control; no enzymes) for 0, 0.5, 1, 2, 4, 6 and 24 hours at 28° C. and 100 rpm agitation compared to percent release of rRFP from rRFP-loaded diatoms incubated in a saline control pH 7 for the duration of the experiment. Data points represent average % protein release of three independent experiments±SEM;

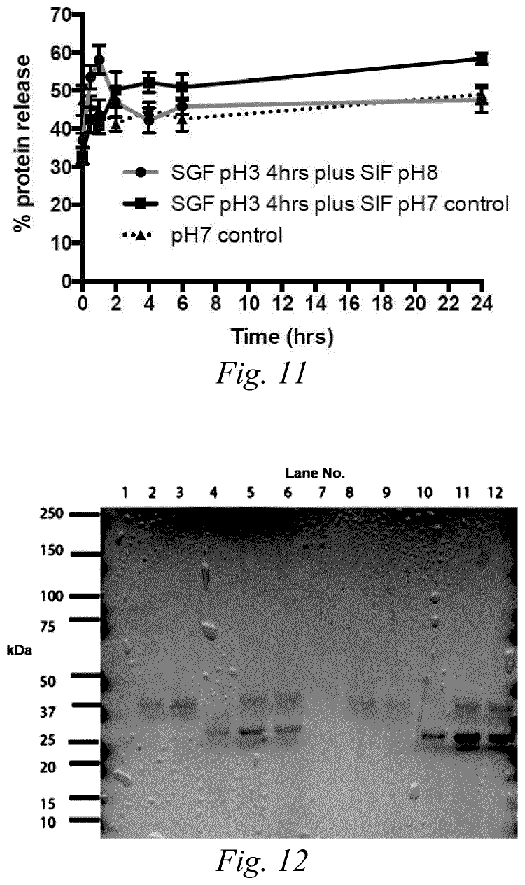

FIG. 11 shows total cumulative percent release of rRFP loaded into diatoms and incubated for 4 hours in SGF pH 3, then in SIF at pH 8 or SIF at pH 7 (control; no enzymes) for 0, 0.5, 1, 2, 4 and 6 hours at 28° C. and 100 rpm agitation compared to percent release of rRFP from rRFP-loaded diatoms incubated in a saline control pH 7 for the duration of the experiment. Data points represent average % protein release of three independent experiments±SEM;

FIG. 12 shows an SDS-PAGE gel illustrating release of rRFP loaded into diatoms and incubated for 4 hours in SGF pH 3, then in SIF at pH 7 or pH 8 for 6 and 24 hours at 28° C. with 100 rpm agitation compared to percent release of rRFP from rRFP-loaded diatoms incubated in a saline control pH 7 for the duration of the experiment. Lanes 1-3: empty diatoms in SIF for 6 hrs; lanes 4-6: rRFP-loaded diatoms in SIF for 6 hrs; lanes 7-9: empty diatoms in SIF for 24 hrs; lanes 10-12: rRFP-loaded diatoms in SIF for 24 hrs; lanes 1, 4, 7 and 10: saline control at pH 7; lanes 2, 5, 8 and 11: SIF at pH 7; lanes 3, 6, 9 and 12: SIF at pH 8; and

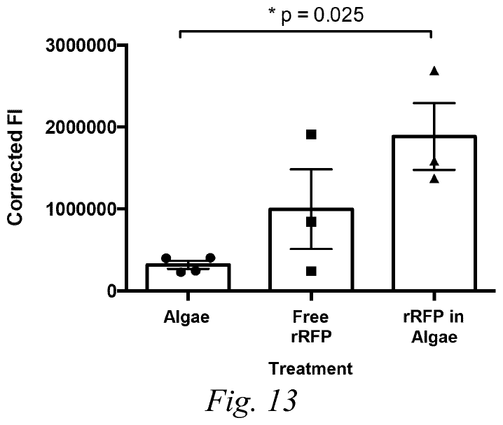

FIG. 13 shows the average corrected fluorescence intensity (±SEM) of adult zebrafish intestine 24 hours after oral gavage with rehydrated algal cells (“Algae”), free rRFP or rehydrated algal cells loaded with rRFP (“rRFP in Algae”). N=3 fish per treatment group and n=4 fish for control group.

### EXAMPLES

### Example 1—Production of Dehydrated Algae

100,000 litres of a culture of Cyclotella meneghiniana was cultured in f/2 Guillard's modified culture medium to a density of around 3 to 3.5 billion cells per millilitre (around 0.15 g per litre).

The composition of the modified f/2 Guillard's culture medium is as follows (per 1000 litres of water): 88.3 g KNO₃; 5.7 g KH₂PO₄; 30 g Na₂SiO₃.5H₂O; 5.0 g EDTA-Na₂; 4.7 g iron (III) chloride (40%); 0.16 g MnSO₄.H₂O; 0.01 g CuSO₄.5H₂O; 0.013 g ZnSO₄; 0.05 ml Chelal®-Co (50 g Co per litre solution); and 0.0063 g Mo (38%).

The algae were then harvested by first filtering through filters having a pore size of 0.2 μm (Liqoflux, Rijen, The Netherlands) at atmospheric pressure, which increased the concentration of algae in the retained suspension by 10-fold.

The retained suspension of algae was then centrifuged using an Evidos 25 (Evodos BV, Raamsdonksveer, The Netherlands), which produced around 100 kg of concentrated algae in the form of a paste comprising around 15% dry material, as measured by a PMB Moisture Analyser (Adam, Milton Keynes UK).

The paste was then freeze-dried in 80×10 kg batches by first transferring the paste to a freeze-drying plate and stored at −20° C. until further processing.

The paste, still frozen on the plate, was removed from the −20° C. storage, and was kept under a vacuum of 0.5 mbar at −20° C. for 36 hours. The paste was then heated from −20° C. to a temperature of 30° C. over a period of 36 hours, still under vacuum.

The process was then stopped, the vacuumed removed and the temperature was allowed to cool to ambient temperature (around 20° C.).

The final freeze-dried product had a moisture content of around 5%, as measured by a PMB Moisture Analyser (Adam, Milton Keynes UK). Other batches had moisture contents of between 2% and 7%. Each 10 kg batch of paste produced around 1.5 kg of freeze-dried algae.

The freeze-dried algae were packaged in heat-sealed packets for ambient storage.

### Example 2—Production of Rehydrated Algae

The freeze-dried algae produced according to Example 1 were rehydrated by adding 1 litre of clean aquaculture water to a standard kitchen blender (Kenwood KM230, 650 W, with blender attachment). The blender was switched on at a moderate mixing speed (speed 4) to agitate the water. Then 20 g or 100 g of freeze-dried algae was added to the water while mixing, and the freeze-dried algae was allowed to blend into the water for 2 minutes. The rehydrated algae were fed to juvenile shrimp.

### Example 3—Production of Rehydrated Algae in Low Water Volume

The freeze dried algae produced according to Example 1 were rehydrated in 50 ml fresh water in a plastic cup and using a magnetic stirrer. The freeze dried algae was added gradually 1 g at a time and stirred at 800 rpm for at least 2 minutes after each addition.

After the addition of 9 g algae, the viscosity of the solution became too high for the magnetic stirrer and mixing continued manually using a plastic spoon.

Cell integrity was observed throughout the experiment using a microscope.

Transition points were at 9 g/50 ml, where the solution turned into a viscous gel and at 17 g/50 ml, where the gel solidified like plasticine. Further rehydration was thus not possible.

### Example 4—Loading of Algae with a Water-Soluble Agent

The freeze-dried algae produced according to Example 1 were loaded with vitamin C as an example of a water-soluble agent.

### Method

A rehydration composition was prepared by dissolving 500 mg per litre vitamin C in filtered natural (North Sea) seawater (20° C.). A second rehydration composition was prepared by dissolving 5 g per litre vitamin C in the seawater. As a control, a third rehydration composition consisted of just the clean seawater without any vitamin C added.

The algae were then rehydrated by adding one of the rehydration compositions to a standard kitchen blender (Kenwood KM230, 650 W, with blender attachment). The blender was switched on at a moderate mixing speed (speed 4) to agitate the rehydration composition. Then 10 g per litre of freeze-dried algae was added to the rehydration composition while mixing, and the freeze-dried algae was allowed to blend into the rehydration composition for 2 minutes. Cell integrity after rehydration was verified using a microscope.

Each of the three rehydration compositions comprising rehydrated algae were split into six replicates. Three replicates of each composition were harvested immediately after rehydration. The remaining three replicates of the treatment groups were incubated in a water bath at 28° C. and provided with moderate aeration. After 6 hours, the remaining three replicates each were harvested.

To remove the rehydration compositions, four 50 ml samples of algal suspension were collected from each replicate in Falcon tubes, and centrifuged at 2,500 rpm for 10 minutes, and the supernatant discarded. The sample as then washed with around 50 ml clean seawater, and centrifuged again. The samples were then washed and centrifuged again. No cell damage was observed from centrifugation.

The supernatant was discarded and the pellet weighed and transferred to Eppendorf tubes. Glass beads were added and the sample homogenized with a bead beater for 60 seconds, broke up the cells completely.

The concentration of vitamin C in the algal cells of the treatment groups and control at the different time points were then analysed by high-performance liquid chromatography with Diode-Array Detection.

### Results

The vitamin C (in μg/g dry weight) found in the algae rehydrated in the different rehydration compositions (plain seawater (control); 500 mg vitamin C per litre; 5,000 mg vitamin C per litre, quantified immediately after rehydration (“Initial”) and after 6 hours incubation time (“6 hours”) is summarized in Table 1 below.

TABLE 1

| Rehydration Composition | Initial | 6 hours |
|---|---|---|
| Seawater | 75.5 ± 32.3 | n.d. |
| 500 mg/l | 94.8 ± 3.3 | 132.3 ± 6.4 |
| 5,000 mg/l | 767.3 ± 160.2 | 731.2 ± 7.1 |

n.d.: not detected

Upon rehydration, algae rehydrated in plain seawater (control) showed a vitamin C level of approximately 75.5 μg/g dry weight. After 6 hours incubation at 28° C. however, no ascorbic acid was present in the algae (below detection limit).

Rehydration in seawater dosed with 500 mg/L ascorbic acid increased the vitamin C level in the algae cells very little (˜95 μg/g dry weight) compared to the control at the initial time point. In contrast to the control however, this level was increased to 132 μg/g dry weight after 6 hours incubation. Rehydration in 5 g/L vitamin C drastically increased the vitamin C level in the algae cells to approximately 770 μg/g dry weight. Moreover this level was maintained for minimum 6 hours.

### Example 5—Loading of Algae with an Amino Acid Glycine, Glucose or Vitamin B1

The freeze-dried algae produced according to Example 1 were loaded with glycine, glucose or vitamin B1 as an examples of water-soluble agents.

### Method

The algae were rehydrated either in distilled water or artificial seawater (i.e. 3.5 g/l NaCl) containing one of glycine, glucose or vitamin B1 (thiamine) to form a solution with a final concentration of 28.4 mM. Algae at 10 g/100 ml of corresponding solutions were slowly added to intensively stirred solutions. As a control, the algae were rehydrated either in distilled water or artificial seawater without glycine, glucose or vitamin B1. The algae were then incubated for 2 hours at 25° C. without stirring

Rehydrated algae were then harvested by removing 2 ml of suspension and centrifuging for 10 min at 13.4 rpm. Received supernatants were discarded and pellets resuspended up to 2 ml in the distilled or sea water and centrifuged for 10 min at 13.4 rpm, the supernatants were discarded and the pellets resuspended up to 2 ml in the distilled or sea water. The operation was repeated 2 times more and the resulting pellet was used to determine the residual levels of glycine, glucose or vitamin B1 in the algae.

To determine the residual levels of glycine, glucose or vitamin B1 in the algae, the cells were destroyed using a pestle and mortar. Microscopic visual analysis demonstrated that over 90% of cells were destroyed with the mortar. Two ml of the product was centrifuged for 15 min at 13.4 rpm. The compounds of interest were measured in the produced supernatants.

The amounts of glycine, glucose or vitamin B1 in the supernatant was determined using regular laboratory chemical or enzymatic methods of assays. Briefly, the levels of amino acids were measured with ninhydrin reagent using glycine standards for calibration. Glucose amounts were measured enzymatically by the Liquick Cor-GLUCOSE commercial kit (Cormay, Poland) with spectrophotometric detection at 540 nm. Glucose concentrations in the samples were estimated using a linear regression of data from a standard curve. Vitamin B1 amounts were evaluated spectrophotometrically (R. O. Hassan and Y. J. Azeez, Tikrit Journal of Pharmaceutical Sciences, 2005, I (2): 1-8).

Results are presented as mean±standard deviation (SD).

### Results

Microscopic visual inspection confirmed that none of the centrifugation regimes described above lead to cell damage.

Table 2 shows the results of measurement of glucose amounts in the final algae preparations. The amount of glucose in algae rehydrated with 28 mM glucose in distilled water was 1.9 fold higher than the amount of glucose in algae rehydrated with distilled water. When the same experiments were carried out in sea water instead of distilled water, the fold-difference was 2.1 times.

TABLE 2
Amounts of glucose (in mg/g dry weight) in algae preparations.
Data are presented as mean ± SD (n = 3).

| Rehydration Conditions | Mean ± SD |
|---|---|
| Distilled water | 0.916 ± 0.075 |
| Distilled water + 28 mM glucose | 1.78 ± 0.25 |
| Sea water | 1.16 ± 0.06 |
| Sea water + 28 mM glucose | 2.46 ± 0.09 |

Table 3 shows the results of measurement of amounts of amino acids in the final algae preparations. The amount of glycine in algae rehydrated with 28 mM glycine in distilled water was 1.3 fold higher than the amount of glycine in algae rehydrated with distilled water. When the same experiments were carried out in sea water instead of distilled water, the fold-difference was 1.9 times

TABLE 3
Amount of amino acids (in mg/g dry weight) in algae preparations.
Data are presented as mean ± SD (n = 3).

| Rehydration Conditions | Mean ± SD |
|---|---|
| Distilled water | 1.99 ± 0.67 |
| Distilled water + 28 mM glycine | 2.69 ± 0.46 |
| Sea water | 1.39 ± 0.49 |
| Sea water + 28 mM glycine | 2.64 ± 0.18 |

Table 4 shows the results of measurement of vitamin B1 in the final algae preparations. The amount of vitamin B1 in algae rehydrated with 28 mM vitamin B1 in distilled water was 10.1 fold higher than the amount of vitamin B1 in algae rehydrated with distilled water. When the same experiments were carried out in sea water instead of distilled water, the fold-difference was 3.9 times

TABLE 4
Amounts of vitamin B1 (thiamine, in mg/g dry
weight) in algae preparations. Data are presented
as mean ± SD (n = 3).

| Rehydration Conditions | Mean ± SD |
|---|---|
| Distilled water | 0.322 ± 0.062 |
| Distilled water + 28 mM thiamine | 3.41 ± 0.21 |
| Sea water | 0.342 ± 0.087 |
| Sea water + 28 mM thiamine | 1.33 ± 0.19 |

### Conclusions

Rehydration of freeze dried algae with solutions of 28 mM glucose or amino acids enriched algae preparations about 2-fold relatively to pure water. Such results were found for experiments using either distilled water or sea water. Vitamin B1 amounts were 4 and 10-fold higher when algae were rehydrated with the solutions of 28 mM vitamin B1 in sea and distilled water respectively.

The tested compounds were retained in the cells even after 3-fold washing with distilled or sea water.

It is expected that any water-soluble agent may be loaded into algae using the method of the invention.

### Example 6—Loading of Algae with Lipid

To assess the ability to load algae with lipids, algae were rehydrated with water supplemented with omega-3 DHA EE fish oil.

In more detail, 3 ml of the oil was transferred into a 15 ml falcon tube. A teaspoon of dehydrated algae powder, made according to Example 1, was added into the tube, and the tube was manually shaken for 2 minutes. Potable water was then added into the tube, up to the 12 ml mark, and the tube was again shaken manually for 2 minutes to form a suspension. The tube containing the suspension was centrifuged for 1 min, and the supernatant was gently removed from the tube without disturbing the pellet.

The pellet was washed by adding potable water to the tube, up to the 12 ml mark, and shaking the tube manually for 2 minutes to form a suspension. The tube containing the suspension was centrifuged for 1 min, and the supernatant was gently removed from the tube without disturbing the pellet. This wash was repeated four times.

The pellet obtained after the fourth wash was resuspended in 8 ml of water and examined under a microscope and the image shown in FIG. 1 was obtained.

As a control, the same procedure was followed using a sample from the same batch of algae, but without the addition of the oil. The image shown in FIG. 2 was obtained.

Comparing FIGS. 1 and 2, it can been seen that oil droplets can be seen within the rehydrated algae that were loaded with oil, and that such droplets are absent from the algae rehydrated without oil.

It is expected that any lipid-soluble agent may be loaded into algae using the present method.

### Example 7—Loading of Algae with Lipid-Soluble Agent

The freeze-dried algae produced according to Example 1 were loaded with astaxanthin as an example of a lipid-soluble agent.

A rehydration composition was prepared by adding 1 g of synthetic astaxanthin to 200 ml of fresh, potable water, then microwaving 3 times for 1 min each time.

The algae were then rehydrated by blending the astaxanthin solution (suspension) together with 1 litre of potable water and 200 mg of the algae powder for 2 minutes.

Microscopic examination of the powder blended with astaxanthin revealed the rehydrated algae cells were strongly ‘coloured’ in red, indicating take-up of the astaxanthin. Astaxanthin particles were able to penetrate inside algae cells and were also absorbed by the porous surface of the siliceous exoskeleton (frustule). It was observed that the association of astaxanthin particles within algae cells remained stable over 24 hours.

### Example 8—Loading of Algae with Recombinant Protein

To determine the loading capacity and efficiency of algae cells for a recombinant protein antigen, algae cells prepared according to Example 1 were loaded with recombinant red fluorescent protein (rRFP), mCherry (˜28 kDa).

In more detail, 25 ml of sterile purified water (Nanopure) containing 0, 0.25, 0.5, 1 or 5 mg of total rRFP was stirred in a glass beaker with a magnetic stirrer at 1,200 rpm. One gram of dehydrated algae powder was added to the central swirl of the stirred mixture. The mixture was then stirred for 4 minutes to form a suspension. A 1-ml subsample was taken from the algae cell suspension and washed twice by centrifugation at 5,000×g for 5 minutes at 4° C. followed by resuspension to the original volume with sterile purified water. Washing removed free rRFP protein that was not taken up by the algae cells.

The washed cells were subsequently lysed with 0.5 mm glass beads (Biospec Products, Inc) in a Tissue Lyser II (Qiagen) for 6 minutes at 28 Hz. The lysed suspension was centrifuged at 5000×g for 5 minutes at 4° C. to pellet cell debris, and 100 μl of the supernatant added in triplicate to a 96 well plate in order to determine the concentration of rRFP that was taken up by the algae cells.

A 1-ml sample from the algae cell suspension before washing was also lysed as detailed above, and the supernatants plated in the 96 well plate in triplicate in order to determine the total concentration of rRFP in the algae suspension of loaded and free rRFP. Loading experiments were performed three independent times for each of the preparations with 0, 0.25, 0.5, 1 or 5 mg of total rRFP to determine reproducibility.

Quantification of rRFP amount was achieved by measuring fluorescence intensity of the samples in triplicate on a Synergy 2 multi-mode plate reader (Biotek) against background controls of cells rehydrated using the same protocol in the absence of rRFP, and correlated to a standard curve of fluorescence intensity versus protein concentration.

The standard reference curve was established by reading the fluorescence in triplicate of 0, 5, 10, 25, 50, 100 and 250 μg/ml rRFP diluted in the 1× rehydrated cell suspension, which was subsequently lysed and the supernatant fluorescence intensity read in triplicate as detailed above.

The loading efficiency and loading capacity of the algae cells were calculated according to the following equations:

$$Loading efficiency = \frac{Concentration of rRFP for washed lysed cells}{Concentration of rRFP for unwashed lysed cells} \times 100 \%$$

$$Loading capacity = \frac{Mass of rRFP for washed lysed cells ( mg )}{Mass of algae ( g )}$$

Recombinant RFP uptake by algae cells was verified by fluorescence imaging, in which loaded cells were washed as detailed above, diluted 1/100 in sterile purified water, transferred to cover glass-bottom 24-well dishes (MatTek, Ashland Mass.), and imaged with an Olympus-FV-1000 laser scanning confocal system. An Olympus IX-81 inverted microscope with an FV1000 laser scanning confocal system (Olympus) was used for confocal imaging. An objective lens with power of 40×/0.75 NA was used. Excitation of rRFP was achieved using 543 nm laser excitation and a suitable excitation/emission optical filter set was used for imaging.

### Results

FIG. 3 shows the loading efficiency (%) of rRFP into diatoms after the addition of 1 g of diatoms to 25 ml of sterile purified water containing 0 to 5 mg total rRFP: i.e., to final rRFP concentrations of 0, 10, 20, 40 and 200 μg/ml. The bars in FIG. 3 represent average loading efficiency of three independent loading experiments±SEM.

FIG. 4 shows the loading capacity (%) of red fluorescent protein (RFP) into diatoms after the addition of 1 g of diatoms to 25 ml of sterile purified water containing 0 to 5 mg total rRFP: i.e., to final rRFP concentrations of 0, 10, 20, 40 and 200 μg/ml. The bars in FIG. 4 represent average loading efficiency of three independent loading experiments±SEM.

Overlays of differential interference contrast (DIC) and confocal fluorescence images of rehydrated and washed algae cells a total rRFP content of 5 mg (concentration of 200 μg/ml) confirmed that fluorescence, and thus rRFP, is localised in intact algae cells.

Thus, recombinant protein can be loaded into algae.

### Example 9—Stability and Release of Recombinant Protein Loaded in Algae Cells in Simulated Fish Gastric and Intestinal Conditions

To determine the potential of using algae for oral delivery of recombinant proteins such as recombinant protein antigens, the stability and release properties algae cells loaded with recombinant RFP was assessed.

In more detail, algae were loaded with 5 mg of rRFP according to Example 8. Two experimental groups, algae loaded with rRFP and free rRFP (rRFP not loaded in algae), were independently incubated with agitation at 100 rpm in simulated gastric fluid (SGF; 0.5% w/v sodium chloride, 0.3% w/v bile salts, 3.2 kU/ml pepsin from porcine gastric mucosa), at pH 2, 3 or 5, at a temperature of 28° C. Control incubations for both experimental groups were carried out in 0.5% w/v sodium chloride at pH 7. Five ml algae suspension samples were centrifuged at 3716×g, washed in purified water and resuspended in 5 ml of the SGF buffer at pH 2, 3 and 5, each in triplicate. At the same time, free rRFP was subjected to the same conditions. One mg rRFP was incubated in 5 ml of the SGF buffer at pH 2, 3 and 5, each in triplicate.

The samples were incubated at 28° C. and agitated at 100 rpm in the dark. To quantify the release and stability of the rRFP within the experimental gastric conditions, subsamples of 500 μl were taken at time 0, 30 minutes, 1 hour, 2 hours, 4 hours, 6 hours and 24 hours. The subsamples were centrifuged at 5000×g for 5 min at 4° C. to pellet the algae cells. The supernatant was evaluated for rRFP release by measuring the rRFP fluorescence intensity, as described in Example 8, in order to estimate release of a recombinant protein antigen prior to reaching the intestine.

The integrity/stability of the rRFP loaded within the algae cells during incubation in SGF for 4 hours at 28° C. was assessed by processing the centrifuged algal pellet at the 4 hour time point to determine the amount/integrity of rRFP still encapsulated within the algae cell. This was quantified as a proportion of the original amount encapsulated within the algal cells at time 0 of addition to SGF. This was compared to free rRFP under the same conditions.

The algae pellets at time 0 and at 4 hours post-incubation were washed once in purified water to remove residual external rRFP prior to lysis of the algae cells with 0.5 mm glass beads (Biospec Products, Inc) in a Tissue Lyser II (Qiagen) for 6 minutes at 28 Hz. The lysed suspension was centrifuged at 5000×g for 5 minutes at 4° C. to pellet cell debris, and 100 μl of the supernatant added in duplicate to a 96 well plate (Nunc) in order to determine the fluorescence intensity as in Example 8.

The integrity/stability of the rRFP released from the algae cells during incubation in SGF at pH 2, 3 and 5 for 4 hours at 28° C. was also assessed by processing the supernatant after centrifugation of the algal cells at the 4 hour time point and running them on an SDS-PAGE to visualize the intensity and degradation of the released rRFP compared to free rRFP under the same conditions.

Fifteen 15 μl subsamples were taken from each treatment group and mixed 1:1 with 2× Laemelli buffer with B-mercaptoethanol (Biorad) followed by debaturation at 95° C. for 5 minutes. Ten μ1 of the samples were loaded per well onto pre-cast 4-15% gradient gels (Biorad). Precision Plus Protein dual colour standards (Biorad) were run on each gel to estimate molecular weight of the proteins. Gels were run at 200 V (100 mA) for 40 minutes, and subsequently stained with coomassie blue (0.25% w/v coomassie brilliant blue 8250, 10% v/v acetic acid, 45% v/v methanol) for 1 hour, followed by de-staining overnight in de-stain solution (10% v/v acetic acid, 45% v/v methanol).

The release and stability of rRFP protein from the algae cells was also evaluated in simulated intestinal conditions in vitro i.e. in simulated intestinal fluid (SIF; 0.5% w/v sodium chloride, 25 U/ml trypsin) at pH 8, following 4 hours of incubation in simulated fish gastric conditions at pH 3 in vitro. This trial was designed to model whether the antigen is released in a stable format within an appropriate time-frame to allow uptake of a recombinant protein by the target animal prior to excretion by defecation. The rRFP-loaded algae cells and algae cells rehydrated in the absence of rRFP were incubated in SGF at pH 3 for 4 hours then incubated in SIF at pH 8, or incubated in SIF without trypsin at pH 7, and samples were taken for analysis after a range of time periods.

Five ml algae suspension samples either loaded or not loaded with rRFP were centrifuged at 3716×g, washed in purified water and re-suspended in 5 ml of pH 3 SGF buffer in triplicates. After 4 hours incubation, the algae cells were centrifuged at 3716×g, washed in purified water and re-suspended in 5 ml of either SIF at pH 8, or SIF without trypsin at pH 7, in triplicate.

A control treatment group in triplicate was also included, in which the rRFP-loaded algae cells or empty algae cells were incubated in 0.5% w/v sodium chloride instead of SGF for 4 hours, followed by incubation in 0.5% w/v sodium chloride rather than SIF for a length of time.

All groups were incubated under gentle agitation of 100 rpm at 28° C. in the dark.

Five-hundred μ1 sub-samples were taken at time periods 0, 30 minutes, 1 hr, 2 hr, 4 hr, 6 hr and 24 hr of incubation. The samples were centrifuged at 5000×g for 5 min at 4° C. to pellet the algae cells. The supernatant was evaluated for rRFP release by fluorescence intensity determination, as described in Example 8, to establish the release of the protein over time within intestinal conditions post-gastric transit. This modelled the availability for uptake in the intestine.

The integrity/stability of the rRFP released from the algae cells during incubation in SIF for 6 and 24 hours at 28° C. was also assessed by processing the supernatant after centrifugation of the algal cells at the 6 and 24 hour time points and analysing the samples by SDS-PAGE to visualize the intensity and degradation of the rRFP compared to rRFP released from algae cells incubated in the SIF pH 7 control or pH 7 control. Fifteen μ1 subsamples were taken from each treatment group and mixed 1:1 with 2× Laemelli buffer with B-mercaptoethanol (Biorad) followed by debaturation at 95° C. for 5 minutes. Ten μ1 of the samples were loaded per well onto pre-cast 4-15% gradient gels (Biorad). Precision plus protein dual color standards (Biorad) were run on each gel to estimate molecular weight of the proteins. Gels were run at 200 V (100 mA) for 40 minutes, and subsequently stained with coomassie blue (0.25% w/v coomassie brilliant blue 8250, 10% v/v acetic acid, 45% v/v methanol) for 1 hour, followed by de-staining overnight in de-stain solution (10% v/v acetic acid, 45% v/v methanol).

### Results

The integrity of the red fluorescent recombinant protein (rRFP), measured by the fluorescence intensity of the protein after 4 hours incubation in the relevant treatment as a percentage of the initial fluorescence intensity at time 0, was significantly higher when the rRFP was loaded into diatoms compared with free rRFP after incubation in SGF at pH 2 (90% vs 75%), pH 3 (94% vs 75%) and pH 5 (95% vs 76%) for 4 hours at 28° C. (FIG. 5).

There was no significant difference between the integrity of the free rRFP and rRFP-loaded into diatoms after 4 hours incubation in a saline control pH 7 at 28° C. (96% and 98%, respectively; FIG. 5).

Therefore, loading the RFP into the diatoms appeared to protect the rRFP from degradation over a 4-hour period in the SGF at pH 2, 3 and 5.

Incubation of rRFP loaded within diatoms in SGF at pH 2, 3 and 5 led to some degradation of the protein released from the diatoms after 4 hours at 28° C. This can be seen in two rRFP degradation products of approximately 24 and 26 kDa, as compared to intact rRFP (˜28 kDa) present in the rRFP released from diatoms in the saline control pH 7 (FIG. 6). However, the free rRFP or its degradation products were found to be completely undetectable by SDS-PAGE after incubation in SGF at pH 2 after 4 hours (FIG. 6; Lane 1) in comparison to rRFP released from diatoms after 4 hours in SGF pH 2, which showed some degradation of the rRFP, but that the rRFP was still present (FIG. 6; Lane 9).

These results in combination suggest that the diatoms significantly enhance stability/integrity of the rRFP loaded within them for at least 4 hours during incubation in SGF, and that the rRFP released from the diatoms within the SGF after 4 hours shows enhanced stability/integrity in comparison to free rRFP.

Recombinant RFP release from the diatoms was detected post-incubation in SGF regardless of the pH, reaching an average of 31%, 32% and 29% release by 6 hours (pH 2, 3 and 5, respectively; FIG. 7). The percent release of the rRFP loaded into the diatoms significantly increased by 24 hours after incubation in SGF at the lower pH of 2 and 3 to an average of 59% and 78%, respectively (FIG. 7). This is likely due to the combination of the pepsin and lower pH enhancing degradation of the diatoms releasing more rRFP.

This suggests that, in vivo, upon feeding on algae comprising recombinant protein such as an antigen, there will be some release of the protein into the stomach, but that the released protein, does not reach high levels unless the algae remain in the stomach conditions at a low pH of 2 or 3 for more than 6 hours, e.g. 24 hours.

The gastric transit time of hybrid tilapia is 4 to 15 hours, and the total gut transit time of Nile Tilapia is around 7 hours. There are reports of 80-90% evacuation of food by 6 to 8 hours in rainbow trout and Nile Tilapia, and Atlantic salmon completely evacuate the entire gut approximately 8 to 24 hours after feeding. Therefore, a timeframe of 6 hours or less for gastric transit in teleost fish is feasible.

The release of rRFP from diatoms in simulated intestinal fluid (SIF) at pH 8 after incubation in SGF at pH 3 for 4 hours was found to be significantly higher after 0.5 (24%) and 1 hour (29%) in comparison to that of diatoms incubated in SIF at pH 7 (control; no enzymes) after incubation in SGF at pH 3 for 4 hours (13% and 11%, respectively), or incubated in a pH 7 saline control for the duration of the treatment (16% and 15%, respectively; FIG. 8). At 2 hours after incubation, the release of rRFP from diatoms in the SIF at pH 7 control (21%) was found to increase significantly above that of the pH 7 saline control (12%) and remained at this level until 24 hours after incubation (29%; FIGS. 8 & 9). The percent release of rRFP from the diatoms incubated in SIF at pH 8 began to decrease from 2 hours (17%) and was found to be the same as that from the diatoms incubated in the pH 7 saline control (19%) out to 24 hours incubation (18%; FIGS. 8 and 9).

The total cumulative release of rRFP from the diatoms, which takes into account the rRFP already released in the prior SGF incubation, shows that the total release of rRFP peaked after 1 hour incubation in SIF at pH 8 (58%) followed by a decline in detectable rRFP by 2 hours (47%), which was maintained until 24 hours after incubation (48%; FIGS. 10 and 11). This may be due to the diatoms releasing the maximum amount of rRFP within a 1-hour incubation in SIF followed by some degradation of the rRFP released within the SIF by trypsin. The percent rRFP release from diatoms incubated in the SIF at pH 7 (control; no enzymes) was found to increase significantly over time after 2 hours and remained at this level until 24 hour after incubation (58%; FIGS. 8 and 9).

### Example 10—Intestinal Uptake of Recombinant Protein Antigen Loaded in Algae in Zebrafish

Intestinal uptake and residence time of free rRFP compared to rRFP loaded in algal cells was quantified using an in vivo adult zebrafish model.

Adult zebrafish were housed in distilled water static tanks at 27° C. with aeration. Fish were starved for 24 hours prior to feeding with algae and/or free rRFP-containing test solutions. The fish were fed by gavage under anaesthesia according to the method of Colleymore et al. (2013, J Vis Exp, 78: 50691) by administering 150 mg/L MS-222 with 250 mg/L sodium bicarbonate, followed by oral gavage of a 5 μl bolus of the test solutions into the anterior lobe via a microcatheter tube. The test solution contained rehydrated algal cells in sterile distilled water, free rRFP in sterile distilled water or algal cells rehydrated with rRFP in distilled water. The rRFP-containing solution each contained the same total amount of rRFP protein.

The fish were allowed to recover after feeding in their housing tanks. The adult zebrafish were euthanized at 24 hours after feeding by administering an overdose of MS-222 (400 mg/L) supplemented with 250 mg/L sodium bicarbonate. The peritoneal cavity was then slit open and the fish fixed in 10% (v/v) neutral-buffered formalin at 4° C. for 2 days.

Fish tissues were cleared via the PACT method of Cronan et al. (2015, Dis. Model Mech., 8: 1643-1650) and Yang et al. (2014, Cell, 158: 1-14). The fish intestines were transferred to 4% acrylamide and 0.25% VA-044 in PBS for 3 days at 4° C. Fish were subsequently transferred to 8% SDS in 200 mM boric acid at pH 8.5 and incubated for 5 days at 37° C., changing the solution every other day. The intestines were washed over a 24 hour period in two washes of PBS with 0.1% Tween-20 at 37° C. The intestines were finally submerged in 80% glycerol in PBS in cover glass-bottom 24-well dishes (MatTek, Ashland, Mass.).

An Olympus IX-81 inverted microscope with an FV1000 laser scanning confocal system (Olympus) was used for confocal imaging. Excitation of rRFP was achieved using 543 nm laser excitation and a suitable excitation/emission optical filter set was used for imaging.

The relative mean corrected fluorescence intensity (CFI) was analysed using FIJI software according to the method of Progatzky et al. (2014, Nat. Commun., 5: 5864) by selecting four equal sized boxes as regions of interest (ROIs) on the intestine and as four background regions (Bkg), and calculating the CFI using the following equation:

CFI_{ROI}=Raw Integrated Density_{ROI}−(Area_{ROI}×Mean Grey Value_{Bkg}).

### Results

Significant levels of rRFP that had been loaded into algal cells was taken up from the intestinal lumen into the intestinal epithelium by 24 hours after feeding (FIG. 13). Smaller amounts of free rRFP was found to be taken up by the intestinal epithelium.

Thus, algae are an effective delivery agent for recombinant proteins.

1. An alga comprising one or more selected from the group consisting of: a therapeutic agent; an exogenous nutritional agent; and an enhanced level of an endogenous nutritional agent.

2. The alga according to claim 1, wherein the alga is a diatom.

3. The alga according to claim 2, wherein the alga belongs to the genus Cyclotella.

4. The alga according to claim 3, wherein the alga is Cyclotella meneghiniana.

5. The alga according to claim 1, wherein the therapeutic agent is selected from the group consisting of: immunogenic agent; antibody; anti-microbial agent; anti-parasitic agent and appetite promoter.

6. The alga according to claim 1, wherein the therapeutic agent or nutritional agent is selected from the group consisting of: protein; carbohydrate; nucleotide; lipid; and steroid.

7. The alga according to claim 1, wherein the therapeutic or nutritional agent is substantially contained within the alga cytoplasm or sub-cellular compartment.

8. The alga according to claim 5, wherein the immunogenic agent is derived from a pathogen.

9. The alga according to claim 5, wherein the immunogenic agent comprises an effective amount of a purified antigen.

10. The alga according to claim 5, wherein the immunogenic agent comprises a nucleotide sequence encoding an antigen.

11. A composition comprising the alga according to claim 1, wherein the composition is included in a diet enhancer, a drug delivery device, a vaccine delivery device and/or an animal feed.

12.-23. (canceled)

24. A method of treatment, comprising administering to an animal the alga comprising a therapeutic agent according to claim 1.

25. (canceled)

26. The method of treatment of claim 24, wherein the animal has a viral, bacterial, or parasitic infection/infestation, and wherein the therapeutic agent is suitable for use in the treatment of a viral, bacterial, or parasitic infection/infestation.
